(19) United States
(12) Patent Application Publication
(10) Pub. No.: US 2006/0127982 A1
(43) Pub. Date: Jun. 15, 2006

(54) Development of an asporogenic bacillus licheniformis and production of keratinase therefrom

(76) Inventor: Jason C.H. Shih, Cary, NC (US)

Correspondence Address: MYERS BIGEL SIBLEY & SAJOVEC, PO BOX 37428, RALEIGH, NC, 27627, US

(21) Appl. No.: 11/261,818

(22) Filed: Oct. 28, 2005

Related U.S. Application Data
(60) Provisional application No. 60/623,759, filed on Oct. 29, 2004.

(51) Int. Cl. C12P 21/06; C12N 9/54; C07H 21/04; C12N 15/74; C12N 1/21

(52) U.S. Cl. 435/69.1; 435/221; 435/252.31; 435/471; 536/23.2

(57) ABSTRACT

A recombinant bacteria and methods of making and using the same are provided. The recombinant bacteria is a recombinant Bacillus having an inactive endogenous spoIIAC gene, with said recombinant Bacillus producing greater quantities of keratinase than a corresponding wild-type Bacillus that does not have said inactive endogenous spoIIAC gene inserted therein. The Bacillus can be Bacillus licheniformis or Bacillus subtilis. The recombinant Bacillus can be a sporulation-deficient Bacillus. A feed additive including keratinase derived from the recombinant Bacillus having an inactive endogenous spoIIAC gene and methods of inactivating an infectious prion protein are also provided.

### CROSS-REFERENCE TO RELATED APPLICATION

This application claims the benefit of U.S. Provisional Application Ser. No. 60/623,759, filed Oct. 29, 2004, the disclosure of which is incorporated by reference herein in its entirety.

### FIELD OF THE INVENTION

The present invention relates to development of a protease and/or sporulation-deficient mutant of a Bacillus species. The present invention further relates to production of keratinase by using such mutant Bacillus strain.

### BACKGROUND OF THE INVENTION

Bacillus licheniformis PWD-1 was first isolated from a poultry waste digester (Williams et al., 1990). It was found to be able to grow on feathers as a primary source of energy, carbon, and sulfur. It also secretes a unique protease, called keratinase, which degrades a wide variety of substrates including chicken feathers (Lin et al., 1992). In addition to promoting the hydrolysis of feather keratin, keratinase is capable of hydrolyzing a broad spectrum of protein substrates, including casein, collagen, elastin, etc., and it displays higher proteolytic activity than most other proteases known in the art.

Keratinase produced from B. licheniformis PWD-1 has a wide-range of applications, including use as a feed supplement to improve bioavailability of nutrients in animal feeds (Odetallah et al., 2003) and in the degradation of the prion protein, the causative agent of mad cow disease (Langeveld et al., 2003).

It has been established that there is a link between sporulation and exoprotease production in Bacillus species. Some protease-deficient mutants show decreased intracellular turnover and also lose the ability to form spores (Mandelstam and Waites, 1968). The reason that protease is needed for sporulation is not well understood. It has been suggested that protease may be responsible for providing the building blocks needed to form new proteins in the prespore. However, Mandelstam and Waites (1968) noted that the protease-minus mutant is not deficient in forming new protein. They also noted that the protease-deficient mutants retained the protein pattern of vegetative cells, whereas wild-type cells (e.g., non-transformed) degrade proteins to form a pattern characteristic of sporulation. Others have reported that a deletion in both the alkaline and neutral proteases had no effect on growth, sporulation, or morphology (Yang et al., 1984). It is now known that mutations in spo0 loci lead to protease-deficient cells because many extracellular enzymes are synthesized during the stationary phase and early sporulation. However, there is evidence that spoIIAC mutations have little effect on proteases in either B. subtilis or B. licheniformis (Coote, 1972; Fleming et al., 1995; Waites et al., 1970).

It is therefore an object of the present invention to provide mutant Bacillus licheniformis strains that are stably defective in sporulation.

It is another object of the present invention to provide mutant Bacillus licheniformis strains that produce keratinase and demonstrate enzyme yield at least equal to that of the wild-type Bacillus licheniformis PWD-1 strain, which may provide methods for commercially feasible mass production of keratinase enzyme that suits the application of crude fermentation product as a feed additive, the destruction of infectious prions and purified fermentation product for biomedical research applications.

### SUMMARY OF THE INVENTION

The present invention relates to development of a protease and/or sporulation-deficient mutant of Bacillus licheniformis PWD-1 (hereinafter, “B. licheniformis WBG strain”). The present invention further relates to production of keratinase by using such mutant B. licheniformis WBG strain.

Additionally, one aspect of the present invention relates to a method of making a keratinase, comprising culturing a recombinant Bacillus in a media, said recombinant Bacillus having an inactive spoIIAC gene, with said recombinant Bacillus producing greater quantities of keratinase than a corresponding wild-type Bacillus that does not have said inactive spoIIAC gene therein; and then collecting said keratinase from said media.

Another aspect of the present invention relates to a recombinant Bacillus having an inactive spoIIAC gene, with said recombinant Bacillus producing greater quantities of keratinase than a corresponding wild-type Bacillus that does not have said inactive spoIIAC gene therein.

A further aspect of the present invention relates to a bacterial culture comprising a recombinant Bacillus, as described herein, in a culture medium.

An additional aspect of the present invention relates to a method of making a recombinant Bacillus, as described herein, comprising the steps of inserting an inactive spoIIAC nucleic acid sequence into an integrative Bacillus expression vector; and then transforming a Bacillus with said integrative Bacillus expression vector.

A further aspect of the present invention relates to a crude, cell-free extract comprised of a mixture of a recombinant Bacillus, as described herein, capable of degrading keratinaceous material.

Another aspect of the present invention relates to a substantially pure, keratinaceous material-degrading, enzyme produced by the recombinant Bacillus described herein.

Yet another aspect of the present invention relates to the keratinase obtained using the methods provided herein in the production of amino acids from a keratinaceous material.

An aspect of the present invention relates to the keratinase obtained using the methods provided herein as a feed additive and/or use in a method of improving animal growth and/or growth performance.

A further aspect of the present invention relates to a method of destroying infective prion protein comprising exposing the infective prion protein to the recombinant Bacillus described herein.

### BRIEF DESCRIPTION OF THE DRAWINGS

FIG. 1. Preparation of mutant AD fragment by Gene SOEing. Four PCR primers (A, B, C, and D) were used in three PCR reactions. Primers B and C contain the 5′ ‘add on’ sequences that allowed one strand from AB to overlap with one strand from CD after AB and CD are mixed, denatured and re-annealed.

FIG. 2. Integration vector pSPO. pSPO was created by ligation of spoIIACΔ to pE194 at the PstI restriction site. eryR includes erythromycin resistance.

FIG. 3. Screening of B. licheniformis PWD-1 transformants by colony PCR. Colonies were analyzed using primers A and D and visualized on 1% agarose gel. Lanes: (M) 100 bp DNA Ladder, (1) PWD-1 (1043 bp), (2) PWD-1 (1043 bp), (3) B. subtilis DB104 transformant (787 bp), (4-6) PWD-1 transformants (787 bp).

FIG. 4. Screening of B. licheniformis PWD-1 clones by colony PCR. Colonies were analyzed using primers A-D and Left-D and visualized on 1% agarose gel. Lanes: (M) 1 kb DNA Ladder, (1) PWD-1/primers A-D(1043 bp), (2) PWD-1/primers Left-D (1505 bp), (3) PWD-1 clone #1/primers A-D (787 bp), (4) PWD-1 clone #1/primers Left-D (1249 bp), (5) PWD-1 clone #2/primers A-D (787 bp), (6) PWD-1 clone #2/primers Left-D (1249 bp), (7) PWD-1 clone #3/primers A-D (787 bp), (8) PWD-1 clone #3/primers Left-D (1249 bp), (9) PWD-1 clone #4/primers A-D (787 bp), (10) PWD-1 clone #4/primers Left-D (1249 bp), (11) PWD-1 clone #5 /primers A-D (787 bp), (12) PWD-1 clone #5/primers Left-D (1249 bp), (13) PWD-1 clone #6/primers A-D (787 bp), (14) PWD-1 clone #6/primers Left-D (1249 bp).

FIG. 5. Heat treatment assay. B. licheniformis PWD-1 and WBG were grown in Schaeffer's sporulation medium for 24 hours, and then heated at 100° C. Samples from various time points were plated onto LB agar for survivors count. (A) Killing curve; (B) survivors on plates after 10 min heat.

FIG. 6. Electron micrographs of B. licheniformis PWD-1 and WBG. (A) A developing spore of PWD-1; (B) WBG sporulation mutant showing the disporic phenotype.

FIG. 7. Comparison of proteolytic, keratinolytic activity and cell growth between asporogenic strain WGB and wild type PWD-1. (●) PWD-1 cell growth; (♦) PWD-1 azocasein assay; (▴) PWD-1 azokeratin assay; (∘) WBG cell growth; (⋄) WBG azocasein assay; (□) WBG azokeratin assay.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs. Further, all publications, U.S. patent applications, U.S. patents and other references cited herein are incorporated by reference in their entireties.

The present invention can be practiced based upon the disclosure described herein, in light of the knowledge of persons skilled in the art, and in light of the information set forth in U.S. Pat. No. 6,743,610, U.S. Pat. No. 6,613,505, U.S. Pat. No. 5,929,221, U.S. Pat. No. 5,712,147, U.S. Pat. No. 5,525,229, U.S. Pat. No. 5,186,961, U.S. Pat. No. 5,171,682, U.S. Pat. No. 5,063,161, U.S. Pat. No. 4,959,311, U.S. Pat. No. 4,908,220, U.S. Patent Application No. 20030108991 (Entitled “Immobilization of Keratinase for Proteolysis and Keratinolysis) and U.S. patent application Ser. No. 10/638,118 (Entitled “Methods and Compositions for Improving Growth of Meat-Type Poultry” the disclosures of all of which are incorporated by reference herein in their entirety.

Except as otherwise indicated, standard methods can be used for the production of viral and non-viral vectors, manipulation of nucleic acid sequences, production of transformed cells, production of proteins and protein fragments by genetic engineering and the like according to the present invention. Such techniques are known to those skilled in the art. See, e.g., “A laboratory manual” Cold Spring Harbor Laboratories, 1982; Ausubel, F. M., et al. (eds.) “Current Protocols in Molecular Biology” John Wiley and Sons, 1995; Harwood, C. R., and Cutting, S. M. (eds.) “Molecular Biological Methods for Bacillus” John Wiley and Sons, 1990), U.S. Pat. No. 4,761,371 to Bell et al. at Col. 6 line 3 to Col. 9 line 65; U.S. Pat. No. 4,877,729 to Clark et al. at Col. 4 line 38 to Col. 7 line 6; U.S. Pat. No. 4,912,038 to Schilling at Col. 3 line 26 to Col. 14 line 12; and U.S. Pat. No. 4,879,224 to Wallner at Col. 6 line 8 to Col. 8 line 59.

In general, recombinant production of Bacilli of the present invention include incorporation of nucleic acid sequences including a deletion of an amino acid sequence associated with sporulation and encoding the nucleic acid product thereof, into a recombinant expression vector in a form suitable for expression of the product in a host cell. “Nucleic acid sequence” as used herein refers to an oligonucleotide, nucleotide, or polynucleotide, and to DNA or RNA of genomic or synthetic origin which may be single- or double-stranded, and represent the sense or antisense strand. Amino acid sequences disclosed herein are presented in the amino to carboxy direction, from left to right. The amino and carboxy groups are not presented in the sequence. Nucleotide sequences are presented herein by single strand only, in the 5′ to 3′ direction, from left to right. Nucleotides and amino acids are represented herein in the manner recommended by the IUPAC-IUB Biochemical Nomenclature Commission, or (for amino acids) by three-letter code, in accordance with 37 C.F.R §1.822 and established usage. See, e.g., Patent In User Manual, 99-102 (November 1990) (U.S. Patent and Trademark Office).

A suitable form for expression provides that the recombinant expression vector includes one or more regulatory sequences operatively-linked to the nucleic acids including the deletion in a manner which allows for transcription of the nucleic acids into mRNA and translation of the mRNA into the protein. Regulatory sequences may include promoters, enhancers and other expression control elements (e.g., polyadenylation signals). Such regulatory sequences are known to those skilled in the art and are described in Goeddel D. D., ed., Gene Expression Technology, Academic Press, San Diego, Calif. (1991). It should be understood that the design of the expression vector may depend on such factors as the choice of the host cell to be transfected and/or the level of expression required. Nucleic acid sequences or expression vectors harboring nucleic acid sequences including the deletion may be introduced into a host cell, which may be of eukaryotic or prokaryotic origin, by standard techniques for transforming cells. As noted above suitable methods for transforming host cells may be found in Sambrook, et al. (Molecular Cloning: A Laboratory Manual, 3rd Edition, Cold Spring Harbor Laboratory Press (2000)) and other laboratory manuals. The number of host cells transformed with a nucleic acid sequence including the deletion will depend, at least in part, upon the type of recombinant expression vector used and the type of transformation technique used. Nucleic acids may be introduced into a host cell transiently, or more typically, for long-term expression of the protein product the nucleic acid sequence is stably integrated into the genome of the host cell or remains as a stable episome in the host cell.

Inactivation of a bacterial gene may involve gene disruption through the insertion or deletion of nucleic acid sequences. In some embodiments, bacterial gene inactivation may take place through insertion of a donor nucleic acid into a target nucleic acid sequence/region of the bacterial genome of interest. The disruption may be targeted or random, and the disrupting donor nucleic acid may be either linear or circular in nature. The donor nucleic acid may comprise but is not limited to homologous sequences to the target and heterologous sequences that may serve as a marker for the inactivation event.

Insertion of a donor nucleic acid sequence into a target nucleic acid sequence may take place through homologous recombination. The homologous recombination event may be a double-crossover integration event with a linear nucleic acid fragment or a single-crossover integration event with a circular nucleic acid. The latter is a circular integration event, wherein a circular donor nucleic acid comprising homologous sequences to target nucleic acid sequences and heterologous nucleic acid sequences recombines with a target region on a bacterial genome. The result is an insertion-duplication recombination event, and the process to accomplish such may be referred to as insertion-duplication mutagenesis.

After a cycle of transformations, the nucleic acid sequence including the deletion of interest is transformed into a species of Bacillus to provide the mutant Bacillus strain.

Keratinase can be obtained by growing the mutant Bacillus which includes nucleic acid sequences encoding a keratinase, under conditions which permit expression of the encoded keratinase, filtering the medium to remove the cells and collecting and concentrating the remaining supernatant by ultrafiltration to obtain the keratinase.

The mutant Bacillus may be cultured under conditions in which the strain grows, and then cultured under conditions which cause the expression of the encoded keratinase, or the cells may be caused to grow and express the encoded keratinase at the same time. Such conditions are well known to one of skill in the art and may vary with the host cell and the amount of enzyme expression level desired. In some embodiments, the medium used to cultivate the transformed host cells may be any medium suitable for keratinase production. The keratinase is recovered from the medium by conventional techniques including separation of the cells from the medium by centrifugation, or filtration, and concentration of the proteins in the supernatant or filtrate by ultrafiltration or evaporation followed by drying via lyophilization or spray-drying. Alternatively, the culture supernatant may be spray-dried or lyophilized after separation without being concentrated. Pure (or substantially pure) keratinases can be obtained by separating the crude keratinase described above into its individual constituents, in accordance with known techniques. See generally W. Jakoby, Ed., Enzyme purification and Related Techniques, Methods in Enzymology, vol. 22 (1971) and vol. 104, pt. C (1984), Academic Press, NY.

Bacterial Strains and Growth Conditions B. licheniformis PWD-1 (ATCC 53575) originally isolated in this lab was used as the wild-type host strain (Williams et al., 1990). B. subtilis DB104 (his nprR2 nprE18 aprΔ3) (Kawamura and Doi, 1984) and Escherichia coli DH5α (Invitrogen, Calif.) were also used as hosts in subcloning. Strains were grown overnight, unless otherwise noted, at 37° C. B. licheniformis PWD-1, a thermophilic bacterium, grows optimally at 50° C. For routine culturing, LB broth or agar (Difco Laboratories) containing appropriate antibiotics was used unless otherwise noted.

DNA Manipulation and Plasmid Construction

In preparation of PCR, genomic DNA from B. licheniformis PWD-1 was isolated using the following method modified from Sambrook et al. (1989). Additionally, many methods for introducing mutations into genes are well known in the art. In one example, a directed mutagenesis can be carried out to provide a modified sequence by the Gene Splicing Overlap Extension method (Molecular Biotechnology, R. M. Horton, 1995, 3, 93-99), which can involve generating a gene sequence in which one or more nucleotides, for example, are introduced or deleted. In one embodiment of the present invention, the primers used for gene splicing by overlap extension (SOEing) are as follows:

A (5′-CCATACTGCAGACGATGGATGAACTGACCGA-3′)
B (5′-TCCTGAACGGTCGGAATCCCAGACCAAACAAGACGCA-3′)
C (5′-TGCGTCTTGTTTGGTCTGGGATTCCGACCGTTCAGGA-3′)
D (5′-AGCCGCTGCAGGATTGCGTCTTGTCTTTG-3′)

The sequences of the primers used were selected from the sequenced B. licheniformis spoIIA operon (NCBI, GI: 304170). Primers B and C would define the deleted region of spoIIAC. The deletion area was selected based upon nucleotide BLAST searches which revealed a high level homology in this region of the gene among related Bacilli. A 256 bp deletion was engineered that spanned nucleotides 141 to 397 of the 767 bp spoIIAC gene. In some embodiments of the present invention, the spoIIAC gene has a less than 300 bp deletion. In particular embodiments, the spoIIAC gene has a 200-300 bp deletion. In some embodiments, the spoIIAC gene has a 256 bp deletion. It should be noted that the 256 bp deletion may encompass amino acids 141 to 397 of a 767 bp spoIIAC nucleic acid sequence. Moreover, it is contemplated that polypeptide and nucleic acid sequences of the present invention encompass those sequences that have at least about 50%, 55%, 60%, 65%, 70%, 75%, 80%, 85%, 90%, 95%, 96%, 97%, 98%, 99% or higher sequence similarity with the sequences specifically disclosed herein (or fragments thereof). As is known in the art, a number of different programs can be used to identify whether a nucleic acid or polypeptide has sequence identity or similarity to a known sequence. Sequence identity and/or similarity can be determined using standard techniques known in the art, including, but not limited to, the local sequence identity algorithm of Smith & Waterman, Ach. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

Primers A and D included PstI restriction sites for use in cloning. Hotmaster Taq polymerase (Eppendorf, AG) was used in PCR reactions and performed as follows: initial denaturation at 94° C., 2 min; denaturation at 94° C. 20sec; annealing at 55° C., 20 sec; extension at 65° C., 1 min; final extension at 65° C., 2 min. The 528 bp AB fragment and 296 bp CD fragment were PCR amplified in separate tubes. PCR products were purified using a PCR Clean-Up Kit (Qiagen, Calif.) as directed by the manufacturer. Products AB and CD were then added as templates for PCR reaction to create AD (787 bp). PCR amplification of the full length AD fragment (1043 bp) without the deletion was performed as a control. The Gene SOEing product, AD, is henceforth referred to as spoIIACΔ.

The purified 787 bp spoIIACΔ fragment was inserted to cloning vector pCR2.1 (Invitrogen) and transformed to E. coli DH5α. Transformants were selected by blue/white screening. Plasmid PCR2.1 carrying spoIIACΔ was digested with PstI to release the spoIIACΔ fragment. PstI-digested spoIIACΔ was gel extracted and purified using Qiagen kits (Qiagen) and subcloned into pE194 (ATTC 37128), creating a new plasmid pSPO. Plasmid pSPO was transformed into B. subtilis DB104 by the competent cell method as previously described (Lin et al., 1997). Transformants were incubated for 90 min at 37° C. and plated on LB containing 20 mg ml⁻¹ erythromycin.

pSPO was transformed into B. licheniformis PWD-1 by modified protoplast transformation (Sanders, 1997). Briefly, cells were grown in LB medium and collected by centrifugation at 6000 g for 10 min. The cells were resuspended in one-tenth volume SMM media (1.0 mole 1⁻¹ sucrose, 0.04 mole 1⁻¹ sodium maleate, 0.04 mole 1⁻¹ MgCl₂.6H₂O, pH 6.5). Lysozyme was added to 2 mg ml⁻¹ and the solution was mixed and incubated for 90 min at 37° C. Protoplasts were collected by centrifugation at 6000 g for 5min and then washed with equal volume SMMP+ media (1.2 g 1⁻¹ Bacto panassay broth, 4% w/v bovine serum albumin in SMM media). The pellet was re-suspended in one-half volume SMMP+. The protoplasts (0.5 mL) was added to a 40 μl mixture containing plasmids in SMM media. The mixture of cells, plasmid, and SMM were immediately transferred to 1.5 ml of 10% w/v polyethyleneglycol for 2 min incubation at 4° C. Cells were then centrifuged at 6000 g, re-suspended in 1 ml SMMP+, and incubated at 37° C. for 90 min. Transformants were plated on regeneration agar (7.0 g 1⁻¹ K₂HPO₄, 3.0 g 1⁻¹ KH₂PO₄, 1.25 g 1⁻¹ (NH₄)₂SO₄, 0.35 g 1⁻¹ MgSO₄.7H₂O, 37.5 g 1⁻¹ HCl, 15 g 1⁻¹ agar) containing 20 μg ml⁻¹ erythromycin. Genetic verification of transformation was accomplished by colony PCR of transformants using primers A and D in the method described above.

Gene Integration and Excision

The method of gene replacement in this experiment takes advantage of the stimulatory effect of rolling-circle replication of thermosensitive plasmids on intramolecular recombination (Biswas et al., 1993; Hamilton et al., 1989). Since plasmid pE194 is naturally temperature sensitive above 42° C. (Noirot et al., 1987), integrants can be selected on the basis of growth at 50° C. Integration of plasmid pE194/spoIIACΔ was accomplished by growing cells at 50° C. in LB broth containing 20 μg ml⁻¹ erythromycin. Those transformants that grew at 50° C. were transferred to LB medium without antibiotics at 37° C. Transformants were continuously transferred to fresh LB medium for 7 generations to select for excision of the plasmid and plasmid cured cells.

The spoIIAC gene amplified from B. licheniformis WBG and PWD-1 was performed using primer sets AD as described above and Left-D (5′-CCAGGAGGATGAGGATGAGC-3′). Sequences were analyzed at Iowa State University (Ames, Iowa) using ABI Model 377 Prism DNA Sequencers.

Heat Treatment Assay

B. licheniformis spores are among the most heat-resistant compared to related Bacilli (Nicholson et al., 2000). To test if the mutant produced spores, a simple heat treatment assay was employed. Cultures of B. licheniformis PWD-1 and WBG were grown in modified Schaeffer's sporulation medium (Schaeffer et al., 1965) for 24 hr at 37° C. to allow sufficient numbers of cells to sporulate. The cultures were boiled for various periods of time and survivors were visualized on LB agar plates.

Determination of Enzyme Activity

Azokeratin assay was used to measure keratinolytic activity as described previously (Lin et al., 1992). Hydrolysis of azocasein was used to determine the proteolytic activity (Wang et al., 2003). In short, 0.2 ml of enzyme aliquot was added to 0.8 ml of 50 mmole 1⁻¹ potassium phosphate buffer (pH 7.5) at 37° C. containing 0.5% w/v azocasein (Sigma, Mo.). The mixture was incubated at 37° C. for 10 min followed by the addition of 0.2 ml of 10% w/v trichloroacetic acid (TCA) to stop the reaction. The supernatant of the mixture was collected by centrifugation and measured by absorbance at 450 nm. The enzymatic activity was determined by the increase of A450 compared with the control.

Electron Microscopy

B. licheniformis PWD-1 and WBG were grown in modified Schaeffer's sporulation medium for 24 hr at 37° C. to allow for sporulation. Cells were harvested by centrifugation at 6000 g, embedded in Spur's resin, and thin-sectioned. Cells were stained with aqueous uranyl acetate for 1 hr and counterstained with lead citrate for 4 min. Samples were then viewed on a JEOL—100 S Transmission Electron Microscope at 30,000× to 35,000× magnification (Electron Microscopy Center, North Carolina State University).

Feed Additive

The keratinase obtained from B. licheniformis WGB using the methods described herein can be included as additive for animal feed. The keratinase should be present in an amount at least sufficient to achieve the intended effect, but the upper limit to the amount of keratinase can be determined based upon achieving the intended effect. In some embodiments, the animal feed comprises from about 0.01% to about 20% B. licheniformis WBG keratinase by weight. Additionally, keratinases used in practicing the present invention can be in crude form or in pure form.

Addition of the B. licheniformis WBG keratinase can increase growth or growth performance of an animal. As used herein, the terms “growth” or “growth performance” refer to increases in either, or both, weight and size (e.g., height, width, diameter, circumference, etc.) over that which would otherwise occur without implementation of the methods and/or administration of the compositions of the present invention. Growth can refer to an increase in the mass (e.g., weight or size) of the entire animal or of a particular tissue (e.g., muscle tissue in general or a specific muscle). Alternatively, growth can indicate a relative increase in the mass of one tissue in relation to another, in particular, an increase in muscle tissue relative to other tissues (e.g., adipose tissue). Growth further relates to nutritional status and disease resistance wherein improvement of nutritional status and/or increase in disease resistance is also indicative of improved growth performance.

In particular, keratinase obtained from B. licheniformis WBG by the methods described herein can be used in a method of growing meat-type poultry, comprising feeding meat-type poultry an animal feed poultry diet wherein the feed further comprises keratinase and is added to the poultry diet in an amount effective to enhance the weight gain of the meat-type poultry. The poultry diet can be an animal feed which includes sources of protein, for example, soybean meal, fish meal, blood meal, poultry by-product (ground poultry offal), meat meal, wheat-meal, rapeseed, canola and combinations of the same. The animal feed further includes carbohydrates, for example, corn, oats, barley, sorghum, or combinations of the same that can be ground into a meal for use in the animal feed. Additionally, the animal feed can include vitamins, minerals, fat, antibiotics, and other substances or compounds as necessary or desired. Non-limiting examples of animal feed poultry diets include cereal-based feeds including cereals such as barley, corn, soya, wheat, triticale, and rye. Corn-soybean, wheat-soybean, and wheat-corn-soybean, sorghum-soybean, and corn-sorghum-soybean represent other non-limiting examples of suitable animal feeds according to the present invention. When the poultry diet is a corn-soybean meal feed, the corn-soybean meal feed comprises from about 60 to about 70% corn by weight and from about 20 to about 30% soybean by weight.

The poultry diet can further be categorized as a starter diet, a grower-type diet, or a finisher-type diet. The precise composition and physical characteristics of the animal feed, and thus the poultry diet, will depend upon the species for which the feed is intended, the age and/or weight of the animal, and the duration of feeding, and can be readily determined by those skilled in the art.

The methods of growing meat-type poultry do not require concurrently providing a specific keratin-containing substrate along with the keratinase. For example, in embodiments of the present invention, the keratinase can directly supplement a poultry diet as a feed additive in contrast to producing a hydrolyzed feather meal as described in Carter, 1998. Thus, the animal feed can be essentially free of keratin (e.g., not more than 1 or 2% by weight keratin.)

Any animal is a suitable subject for increasing growth as described herein, including cows, sheep, pigs, cats, dogs, ferrets, and avians. Suitable subjects can be of any age range including neonatal animals, developing animals, and mature animals. In some embodiments, the suitable subject can be an avian, such as a chicken, and particularly a broiler chick. In other embodiments, the suitable subject can be an immature, developing, or mature bird. In other embodiments, the suitable subject can be a chicken that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 days old, or within any range of these numbers. Thus, the present invention provides a variety of different feeds, including pet feed, poultry feed, and pig feed.

The animal feed supplement of the present invention can also enable a conventional animal feed to be modified by reducing its energy, and/or protein, and/or amino acid content while simultaneously maintaining the same nutritional levels of energy, protein, and amino acids available to the animal. Consequently, the amounts of costly energy and protein supplements typically included in an animal feed can be reduced as compared to conventional feeds.

Destruction of Infectious Prion Proteins

As noted in U.S. Pat. No. 6,613,515, incorporated by reference specifically herein, Prion proteins are conformationally anomalous proteins that are associated with infectious neurodegenerative diseases in human as well as non-human mammalian species.

Prion diseases in non-human mammalian species include scrapie (sheep), transmissible mink encepohalopathy (minks), chronic wasting disease (elk, deer), bovine spongiform encephalopathy (BSE) (cows), feline spongiform encephalopathy (cats), and simian spongiform encephalopathy (monkeys).

In humans, a variety of neurodegenerative conditions are etiologically associated with prion proteins, including Creutzfeldt-Jakob disease, Gerstmann-Straussler-Scheinker syndrome, fatal insomnia, kuru, and variant Creutzfeldt-Jakob disease. Pathogenesis of human prion diseases is associated with carnivorism (BSE-infected beef, causing new variant Creutzfeldt-Jakob disease), administration of human growth hormone (causing iatrogenic Creutzfeldt-Jakob disease) and ritualistic cannibalism (causing kuru).

Infectious prion proteins are resistant to destruction by conventional methods that denature and otherwise degrade conformationally normal proteins, including methods such as autoclaving (even temperatures as high as 200° C. are not effective to inactivate infectious prion proteins), boiling, freezing, and exposure to reagents such as formaldehyde, carbolic acid and chloroform. Typically, incineration or treatment with bleach is employed to destroy the pathogenic isoform of the prion protein.

It therefore would be an advance in the art to provide a composition and methodology for destruction of infectious prion proteins applicable to the treatment of biological materials, e.g., animal tissue containing or contaminated with infectious prion proteins.

Embodiments of the present invention provide methods of treatment of tissue associated with, i.e., containing or contaminated with, infectious prion protein, enhancing degradability of an infectious prion protein by proteolytic enzymatic degradation treatment, removing infective prion protein from mammalian tissue containing or contaminated with same, degrading and/or partially degrading infectious prion protein and/or processing an animal meat product or by-product to clear BSE-mediating infectious prion protein therefrom. The methods include contacting the infective prion protein or material containing the same with a keratinase derived from a mutant Bacillus of the present invention. The method may further include (a) heating a tissue to a sufficient temperature and for sufficient time to enhance the proteolytic degradability of infective prion protein associated with the tissue; and (b) exposing the heated tissue to a proteolytic enzyme that is effective for at least partial reduction of infective prion protein associated with such tissue. Further methods of destroying infective prion protein applicable to the methods provided herein are disclosed in U.S. Pat. No. 6,613,515.

The present invention is explained in greater detail in the following non-limiting examples.

### EXAMPLE 1

### Construction of Integration Vector pSPO

The PCR reactions which included the 528 bp amplified with the AB primer set (AB) and the 296 bp amplified with the CD primer set (CD) were performed separately.

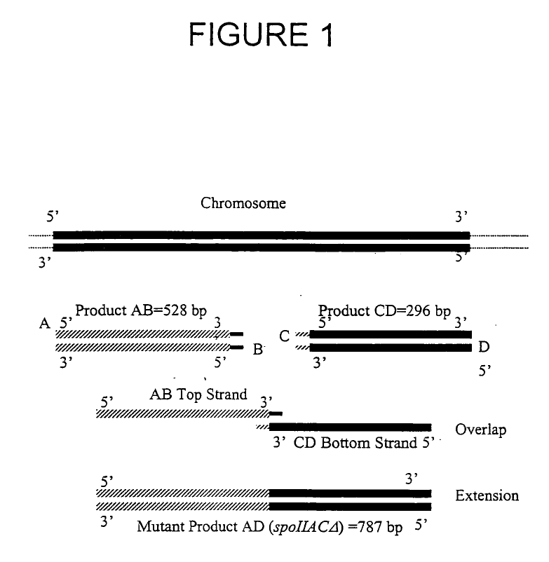

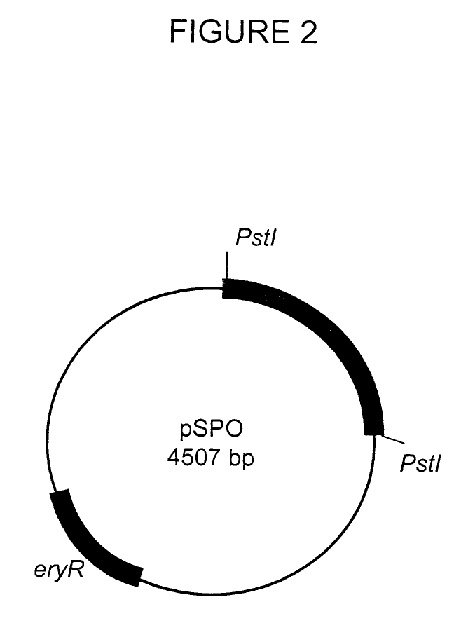

Each product was used as the template for the SOEing reaction generating product AD (FIG. 1). The expected 787 bp size of AD was gel extracted and purified. Purified AD (spoIIACΔ) was ligated to vector pCR2.1 (Invitrogen) and transformed into E. coli DH5α. Transformants were initially selected by blue/white screening and further examined by restriction digestion of isolated plasmids. The 771 bp spoIIACΔ insert was digested from the plasmid by PstI and subcloned into plasmid pE194, generating plasmid pSPO (FIG. 2).

### EXAMPLE 2

### The spoIIACΔ Gene Replacement in B. licheniformis PWD-1

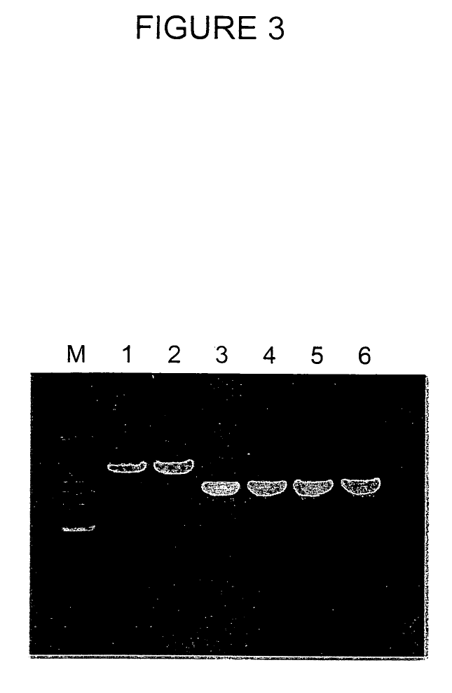

B. licheniformis PWD-1 transformants were selected on regeneration agar containing erythromycin. Colony PCR using primers A and D was performed to verify the transformants (FIG. 3). A single positive transformant was streaked onto LB plates containing erythromycin and grown at 50° C. Survivors were presumed to be integrants. Integrants were inoculated in LB medium without antibiotics at 37° C. This was repeated to select for excision of the plasmid and for plasmid curing.

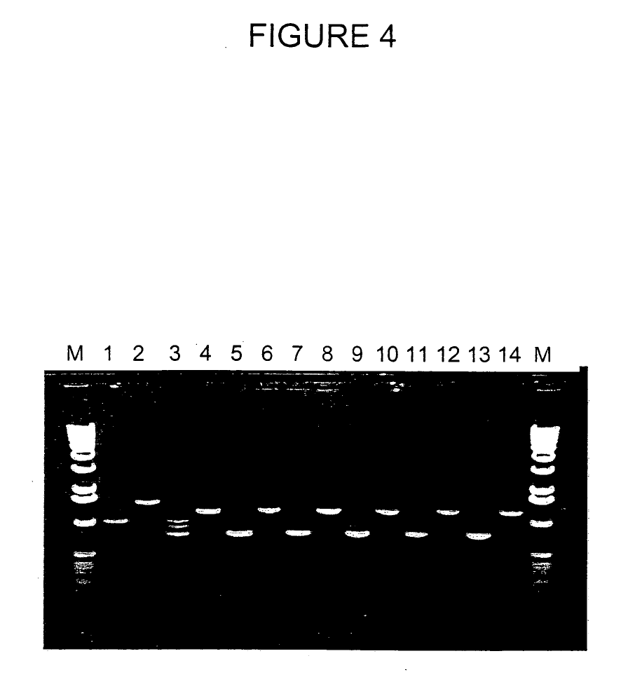

Gene replacement occurring in PWD-1 was verified by PCR analysis. To confirm that the deletion was chromosomally based and not plasmid-borne, a fifth primer Left-D as described above was introduced. Clones were tested for integration/excision by PCR using primer sets A-D and Left-D and visualized on a 1% agarose gel (FIG. 4). The sequence of Left-D is not found in plasmid pSPO, therefore successful amplification must be chromosomally based. The primer was designed 462 bp upstream from primer A and intersects the start codon of spoIIAΔ. The expected size fragments for the mutation with primers A-D and Left-D were 787 bp and 1249 bp, respectively. Five clones produced the expected sizes of fragments (Clones #2-6), while one (Clone #1) produced intermediate products of the wild-type and mutant spoIIACΔ gene. Clone #6 was selected and named B. licheniformis WBG.

### EXAMPLE 3

### Heat Treatment Assay and Electron Microscopy

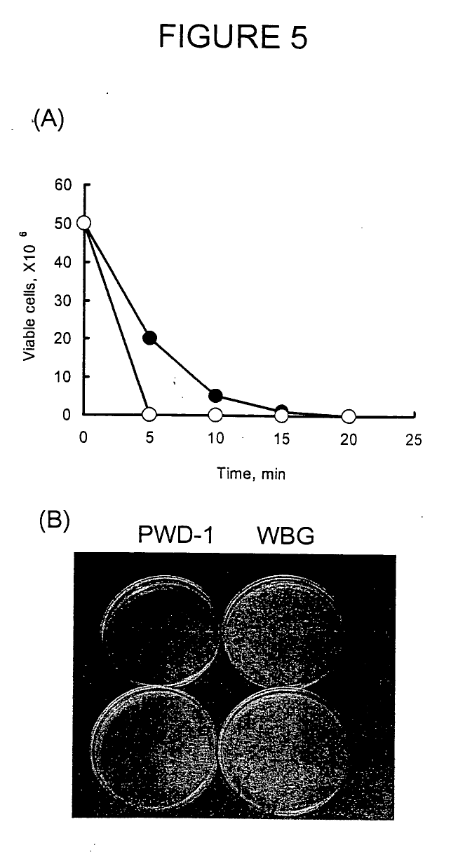

The heat treatment assay is a simple and commonly used method to infer the presence or absence of spores. In this investigation, B. licheniformis WBG was shown to lose heat resistance compared to the wild-type PWD-1 (FIG. 5). No survivors were detected after 5 min of boiling with repeated tests. In contrast, the highly heat-resistance spores of B. licheniformis PWD-1 could be detected after 20 min heating. Their presence was inferred by the ability to grow after treatment.

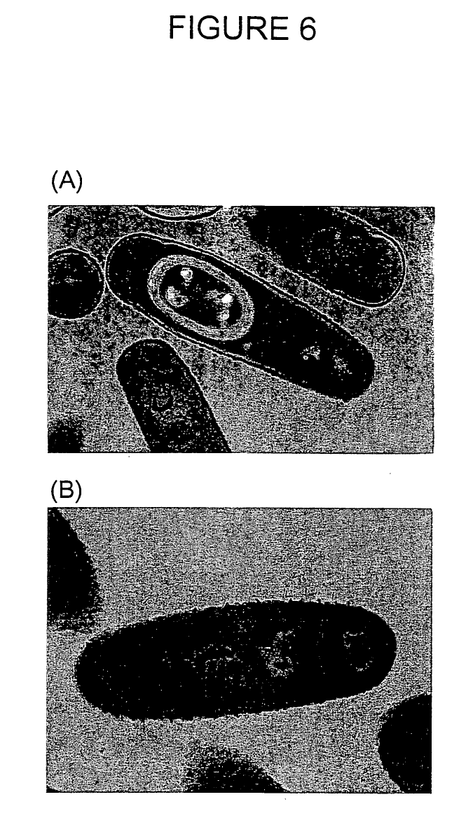

The use of electron microscopy further confirmed the fact that the mutant was asporogenic. No spores were detected among many thin-sectioned cells in the mutant. Many of the cells, like the one shown in FIG. 6B, were clearly disporic, as expected. Other cells of the mutant strain were dividing by binary fission or were not in the process of division at all. However, no cells were observed that progressed beyond stage II (septation) of sporulation. When B. licheniformis PWD-1 was examined, all the morphological stages of sporulation could be observed. Mature endospores from B. licheniformis PWD-1 are shown in FIG. 6A.

### EXAMPLE 4

### Keratinase Production

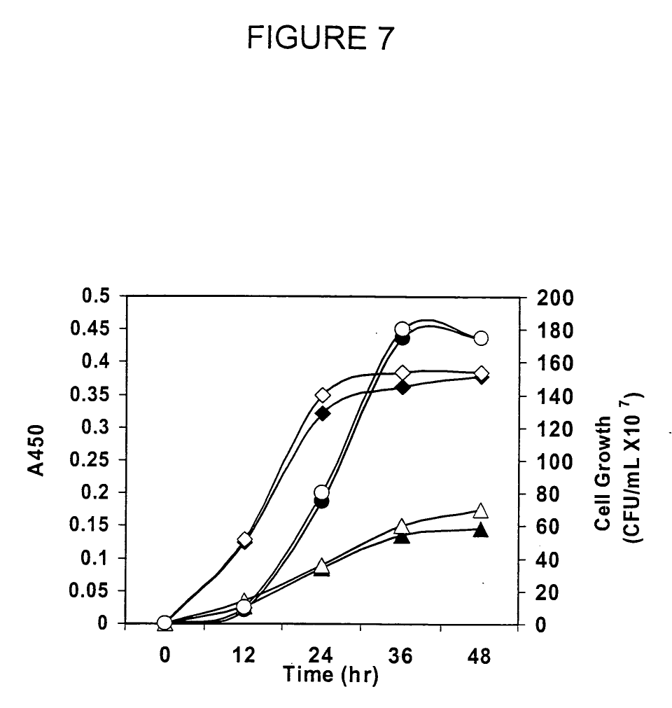

The results of the azokeratin and azocasein assays are shown in FIG. 7. B. licheniformis WBG was found to secrete comparable levels of protease with B. licheniformis PWD-1. The data suggest that the mutation do not suppress keratinase production. Cell growth was also similar between two strains. Moreover, a goal of this study was to develop a sporulation-deficient strain of B. licheniformis PWD-1 and characterize its phenotype of keratinase synthesis and secretion. B. licheniformis WBG was successfully created using the Splicing by Overlap Extension PCR and thermosensitive integration vector pE194. This method allowed the creation of a precise 256 bp deletion of spoIIAC in the chromosome of B. licheniformis, as verified by PCR and DNA sequencing.

Results of this study correlate with those of Fleming et al.(Fleming et al., 1995). However, the deletion reported here is smaller and was demonstrated to be sufficient to disrupt spoIIAC transcription. The gene replacement strategy using pE194 was also similar. The present study used a PCR primer upstream (Primer Left-D) of the deletion to confirm gene replacement. The use of this upstream primer was simpler and useful in confirming gene replacement.

The σF mutant is totally asporogenic as confirmed by the heat treatment assay and electron micrographs. The disporic phenotype observed is consistent with previous studies of B. licheniformis and B. subtilis spoIIA mutants (Fleming et al., 1995).

The mutation does not appear to have any significant detrimental effect on cell growth or production of keratinase. In fact both azokeratin and azocasein assays reveal that the mutant produces more protease than does wild-type PWD-1. This was an unexpected result, as σF mutants of B. licheniformis were shown to produce nearly identical amounts of protease by Fleming et al. (1995). However, it is possible that increased σH accumulation in the mutant is responsible for the increase in protease.

There is no evidence of suppressor mutations, and the chance of reversion with such a large deletion should be minimal. In summary, the method employed in this study created an asporogenic mutant that retains important industrial capabilities, such as the production of keratinase.

The keratinase enzyme produced according to the methods described hereinabove can be supplemented in animal feed as a feed additive, in a manner that improves the digestibility and nutritional value of such feed or in the treatment of infectious prion-related disease.

### REFERENCES

Biswas, I., Gruss, A., Ehrlich, S. D. and Maguin, E. (1993) High-efficiency gene inactivation and replacement system for gram-positive bacteria. Journal of Bacteriology 175, 3628-3635.
Coote, J. G. (1972) Sporulation in Bacillus subtilis. Characterization of oligosporogenous mutants and comparison of their phenotypes with those of asporogenous mutants. J Gen Microbiol 71, 1-15.
Fleming, A. B., Tangney, M., Jorgensen, P. L., Diderichsen, B. and Priest, F. G. (1995) Extracellular enzyme synthesis in a sporulation-deficient strain of Bacillus licheniformis. Applied and Environmental Microbiology 61, 3775-3780.
Hamilton, C. M., Aldea, M., Washburn, B. K., Babitzke, P. and Kushner, S. R. (1989) New method for generating deletions and gene replacements in Escherichia coli. Journal of Bacteriology 171, 4617-4622.
Kawamura, F. and Doi, R. H. (1984) Construction of a Bacillus subtilis double mutant deficient in extracellular alkaline and neutral proteases. Journal of Bacteriology 160, 442-444.
Langeveld, J. P., Wang, J. J., Van de Wiel, D. F., Shih, G. C., Garssen, G. J., Bossers, A. and Shih, J. C. (2003) Enzymatic degradation of prion protein in brain stem from infected cattle and sheep. Journal of Infectious Diseases 188, 1782-1789.
Lin, X., Lee, C. G., Casale, E. S. and Shih, J. C. H. (1992) Purification and characterization of a keratinase from a feather-degrading Bacillus licheniformis strain. Applied and Environmental Microbiology 58, 3271-3275.
Lin, X., Wong, S. L., Miller, E. S. and Shih, J. C. (1997) Expression of the Bacillus licheniformis PWD-1 keratinase gene in B. subtilis. Journal of Industrial Microbiology & Biotechnology 19, 134-138.
Mandelstam, J. and Waites, W. M. (1968) Sporulation in Bacillus subtilis. The role of exoprotease. The Biochemical Journal 109, 793-801.
Nicholson, W. L., Munakata, N., Horneck, G., Melosh, H. J. and Setlow, P. (2000) Resistance of Bacillus endospores to extreme terrestrial and extraterrestrial environments. Microbiology and Molecular Biology Review 64, 548-572.
Noirot, P., Petit, M. A. and Ehrlich, S. D. (1987) Plasmid replication stimulates DNA recombination in Bacillus subtilis. Journal of Molecular Biology 196, 39-48.
Odetallah, N. H., Wang, J. J., Garlich, J. D. and Shih, J. C. (2003) Keratinase in starter diets improves growth of broiler chicks. Poultry Science 82, 664-670.
Sanders, J. P. M., J. A. van den Berg, P. M. Andreoli, B. Kortrijk, Y. J. Vos, J. H. van Ee, J. S. M. Mulleners. (1997) Transformed industrial Bacillus strains and methods for making and using them., U.S. Pat. No. 5,624,829.
Schaeffer, P., Millet, J. and Aubert, J. P. (1965) Catabolic repression of bacterial sporulation. Proceedings of the National Academy of Sciences of the United States of America 54, 704-711.
Waites, W. M., Kay, D., Dawes, I. W., Wood, D. A., Warren, S. C. and Mandelstam, J. (1970) Sporulation in Bacillus sutbtilis. Correlation of biochemical events with morphological changes in asporogenous mutants. The Biochemical Journal 118, 667-676.
Wang, J. J., Swaisgood, H. E. and Shih, J. C. H. (2003) Production and characterization of bio-immobilized keratinase in proteolysis and keratinolysis. Enzyme and Microbial Technology 32, 812-819.
Williams, C. M., Richter, C. S., Mackenzie, J. M. and Shih, J. C. H. (1990) Isolation, identification, and characterization of feather-degrading bacterium. Applied and Environmental Microbiology 56, 1509-1515.
Yang, M. Y., Ferrari, E. and Henner, D. J. (1984) Cloning of the neutral protease gene of Bacillus subtilis and the use of the cloned gene to create an in vitro-derived deletion mutation. Journal of Bacteriology 160, 15-21.

Although the invention has been described with respect to various illustrative embodiments, features and aspects, it will be appreciated that the utility of the invention is not thus limited, but rather extends to and includes various other modifications, alterations and other embodiments, as will readily suggest themselves to those of ordinary skill in the art based on the disclosure herein. The invention is therefore intended to be broadly construed, as encompassing all such modifications, alterations and other embodiments within the spirit and scope of the ensuing claims.

That which is claimed:

1. A method of making a keratinase, comprising:
(a) culturing a recombinant Bacillus in a media, said recombinant Bacillus having an inactive endogenous spoIIAC gene, with said recombinant Bacillus producing greater quantities of keratinase than a corresponding wild-type Bacillus that does not have said inactive endogenous spoIIAC gene therein; and then
(b) collecting said keratinase from said media.

2. The method of claim 1, wherein said Bacillus is selected from the group consisting of Bacillus licheniformis and Bacillus subtilis.

3. The method of claim 1, wherein said Bacillus is Bacillus licheniformis.

4. The method of claim 1, wherein said recombinant Bacillus is a protease-deficient Bacillus.

5. The method of claim 1, wherein said recombinant Bacillus is a sporulation-deficient Bacillus.

6. The method of claim 1, wherein said recombinant Bacillus is not resistant to erythromycin.

7. The method of claim 1, wherein said corresponding wild-type Bacillus is Bacillus licheniformis PWD-1 or Bacillus subtilis DB 104.

8. The method of claim 1, wherein said spoIIAC gene has a less than 300 bp deletion.

9. The method of claim 1, wherein said spoIIAC gene has a 200-300 bp deletion.

10. The method of claim 1, wherein said spoIIAC gene has a 256 bp deletion.

11. The method of claim 10, wherein said 256 bp deletion encompasses amino acids 141 to 397 of a 767 bp spoIIAC nucleic acid sequence.

12. The method of claim 1, wherein said recombinant Bacillus produces at least 30 mg more keratinase per 10⁶ cells in 48 hours as compared to wild-type Bacillus in the same culture condition.

13. A recombinant Bacillus having an inactive endogenous spoIIAC gene, with said recombinant Bacillus producing greater quantities of keratinase than a corresponding wild-type Bacillus that does not have said inactive endogenous spoIIAC gene therein.

14. The recombinant Bacillus of claim 13, wherein said Bacillus is selected from the group consisting of Bacillus licheniformis and Bacillus subtilis.

15. The recombinant Bacillus of claim 13, wherein said Bacillus is Bacillus licheniformis.

16. The recombinant Bacillus of claim 13, wherein said recombinant Bacillus is a protease-deficient Bacillus.

17. The recombinant Bacillus of claim 13, wherein said recombinant Bacillus is a sporulation-deficient Bacillus.

18. The recombinant Bacillus of claim 13, wherein said recombinant Bacillus is not resistant to erythromycin.

19. The recombinant Bacillus of claim 13, wherein said corresponding wild-type Bacillus is Bacillus licheniformis PWD-1 or Bacillus subtilis DB104.

20. The recombinant Bacillus of claim 13, wherein said spoIIAC gene has a less than 300 bp deletion.

21. The recombinant Bacillus of claim 13, wherein said spoIIAC gene has a 150-350 bp deletion.

22. The recombinant Bacillus of claim 21, wherein said 150-350 bp deletion encompasses amino acids 150 to 200 of a 767 bp spoIIAC nucleic acid sequence.

23. The recombinant Bacillus of claim 21, wherein said 150-350 bp deletion encompasses amino acids 200 to 250 of a 767 bp spoIIAC nucleic acid sequence.

24. The recombinant Bacillus of claim 21, wherein said 150-350 bp deletion encompasses amino acids 250 to 300 of a 767 bp spoIIAC nucleic acid sequence.

25. The recombinant Bacillus of claim 21, wherein said 150-350 bp deletion encompasses amino acids 300 to 350 of a 767 bp spoIIAC nucleic acid sequence.

26. The recombinant Bacillus of claim 13, wherein said spoIIAC gene has a 200-300 bp deletion.

27. The recombinant Bacillus of claim 13, wherein said spoIIAC gene has a 256 bp deletion.

28. The recombinant Bacillus of claim 27, wherein said 256 bp deletion encompasses amino acids 141 to 397 of a 767 bp spoIIAC nucleic acid sequence.

29. A bacterial culture comprising a recombinant Bacillus of claim 13 in a culture media.

30. A method of making a recombinant Bacillus of claim 13, comprising the steps of:
(a) inserting an inactive spoIIAC nucleic acid sequence into an integrative Bacillus expression vector; and then
(b) transforming a Bacillus with said integrative Bacillus expression vector.

31. The method of claim 30, wherein said integrative Bacillus expression vector is a plasmid vector.
